(19) United States
(12) Patent Application Publication
(10) Pub. No.: US 2008/0139984 A1
(43) Pub. Date: Jun. 12, 2008

(54) Corset to relieve the cervical spine

(76) Inventor: Vinko Tranfic, Split (HR)

Correspondence Address: KATHLEEN T. PETRICH, GRAHAM & DUNN PC, PIER 70 2801, ALASKAN WAY , SUITE 300, SEATTLE, WA, 98121-1128, US

(21) Appl. No.: 12/012,842

(22) Filed: Feb. 5, 2008

(30) Foreign Application Priority Data
Aug. 12, 2005 (HR) PCT/HR05/00043

(51) Int. Cl. A61F 5/00; A42B 5/00; A42B 1/24; A42B 1/00

(52) U.S. Cl. 602/18; 602/19; 2/207; 2/209.13; 2/195.1

(57) ABSTRACT

The corset to relieve the cervical spine includes the head strap (1) and the waist strap (5). The head strap is fixed over the forehead and the waist strap around the body. The head and the waist straps are mutually connected with strips that can be adjusted to the height of the person using the corset. The strips are to limit bending of the head forward and to the left and right, as well as its rotation around the vertical axis. This produces a significant fixation that enhances effects of eccentric contraction of the deep neck muscles. This, in turn, at head bending or rotation, significantly diminishes aggravating effects to the cervical spine. With regard to the strip construction, there are three corset embodiments: corset (A) with buckles (10) on strips, corset (B) with holes (15) on strips, and corset (C) with buckles and holes on strips. The strips may be connected to a cap or head scarf instead of the head strap.

### TECHNICAL FIELD

The present invention relates generally to corsets that relieve the cervical spine. In particular, the present invention relates to a corset that comprises a head strap and a waist strap, mutually connected with strips that limit head bending and rotation. In the International Patent Classification it is classified as A—Human necessities; A 41—Wearing apparel; A 41 C—Corsets; 1/00—Corsets or girdles.

### BACKGROUND OF THE INVENTION

Corsets that relive the spine have been well-known for years. Spine diseases are among the most common ones. Body movements and irregular postures in daily activities often overload the spine. Such movements and postures are to be avoided or limited to the minimum. The deep muscles of the neck are important because they, by eccentric contractions, prevent the neck from bending forward and sideward and when the head is rotated. By their activity they particularly protect the neck lordosis, that is, forward curvature of the neck, thus protecting this fragile part of the spine. This way, musculature helps the disc and ligament soft tissues to absorb stressful situations. Unfortunately, a large part of our daily activities include head movements and forward positions, sideward positions, and rotated positions, where there are greater dangers of biomechanical damage to the discs. Having this problem in mind, a corset is to be designed to enhance effects of the useful neck musculature and to limit forward, sideward and rotated head movements.

Today there are numerous soft collars that fix the cervical spine. Also there are plastic collars that limit neck movements. In the state of the art there are two known patent documents that are particularly relevant: U.S. Pat. No. 5,336,139 and U.S. Pat. No. 3,957,040, relating to the limitation of head inclination and rotation. Although by their construction they are close to this invention, they do differ in essential details. This particularly relates to flexibility and complexity of embodiment stated in the referral documents.

### SUMMARY OF THE INVENTION

The present invention is directed to corsets that relieve the cervical spine, comprising the head strap and the waist strap, mutually connected with one strip to limit forward movement of the head, two strips to limit sideward movements and two strips to limit head rotation around the vertical axis. The strip length is adjustable to fit the height of the person wearing it. By their construction, there are three corset embodiments: corset with strips and adjustable buckles, corset with strips and holes, and corset with strips, buckles, and holes.

The corset that relieves the cervical spine can be used:
in physical medicine;
for educational purposes (learning of proper movements);
prevention in sports (rowing, sailing, tennis, basketball, etc.); and
in performing daily actions and other actions that require forward bending of the cervical spine, since it releases the tender, soft tissues of the neck and protects them from injuries.

These and other advantages will become more apparent upon review of the Drawings, the Detailed Description of the Invention, and the Claims.

### BRIEF DESCRIPTION OF THE DRAWINGS

Like reference numerals are used to designate like parts throughout the several views of the drawings, wherein:

FIG. 1 is a back perspective view illustrating a corset with strips and buckles of embodiment A;

FIG. 2 is a back perspective view illustrating a corset with strips and holes of embodiment B;

FIG. 3 is a back perspective view illustrating a corset with strips, buckles, and holes of embodiment C;

FIG. 4 is a back perspective view illustrating the corset shown in FIG. 1 on the user's body;

FIG. 5 is a side perspective view illustrating a partial section of the corset with strips attached to a cap that acts as the head strap;

FIG. 6 is a side perspective view illustrating a partial section of the corset with strips attached to a scarf that acts as the head strap; and

FIG. 7 is a back perspective view illustrating the scarf corset on the user's body.

### DETAILED DESCRIPTION OF THE INVENTION

The corset to relieve the cervical spine, according to this invention, comprises: the head strap, the waist strap, the strips that limit forward bending of the head, the strips that limit sideward bending of the head, and the strips that limit head rotation around the vertical axis. The head strap can be used directly or as part of a cap or head scarf.

By construction of the strips, there are three embodiments of the corset to relieve the cervical spine:
corset A, with strips and buckles;
corset B, with strips and holes; and
corset C, with strips, buckles, and holes.

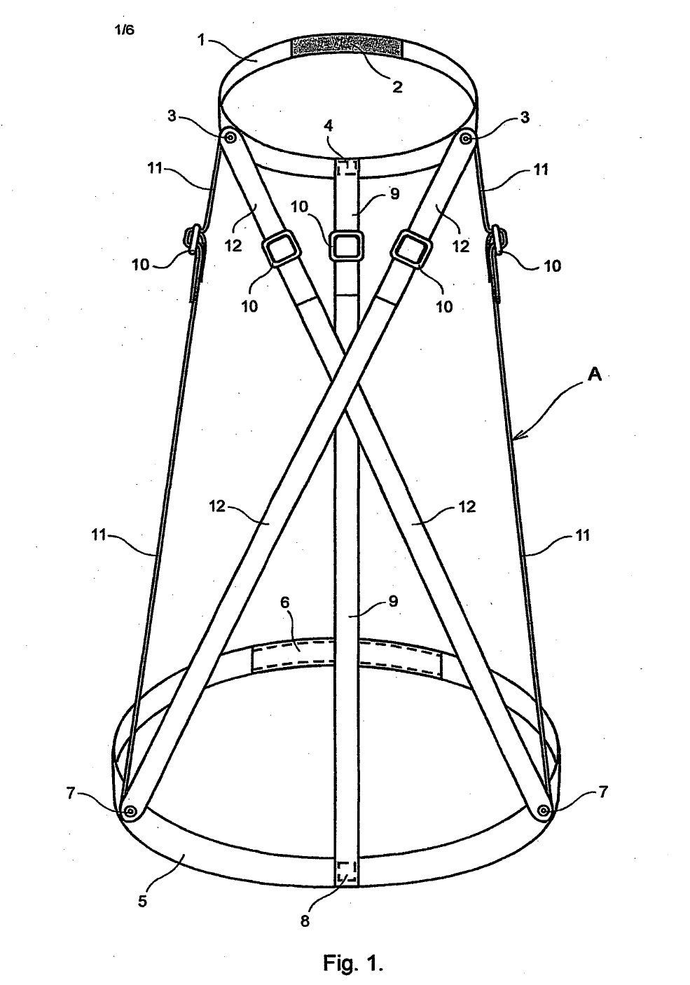

### Corset A, FIG. 1, Comprises:

head strap 1, with fasteners 3, for detaching and connecting strips 11 and 12;
waist strap 5, with fasteners 7, for detaching and connecting strips 11 and 12;
strip 9, with buckle 10 that is in points 4 and 8, firmly fixed to straps 1 and 6;
strips 11 and 12, with buckles 10, movably fixed to fasteners 3 and 7; and
strips 12, with buckles 10, crossed and detachably fixed to fasteners 3 and 7 over the back.

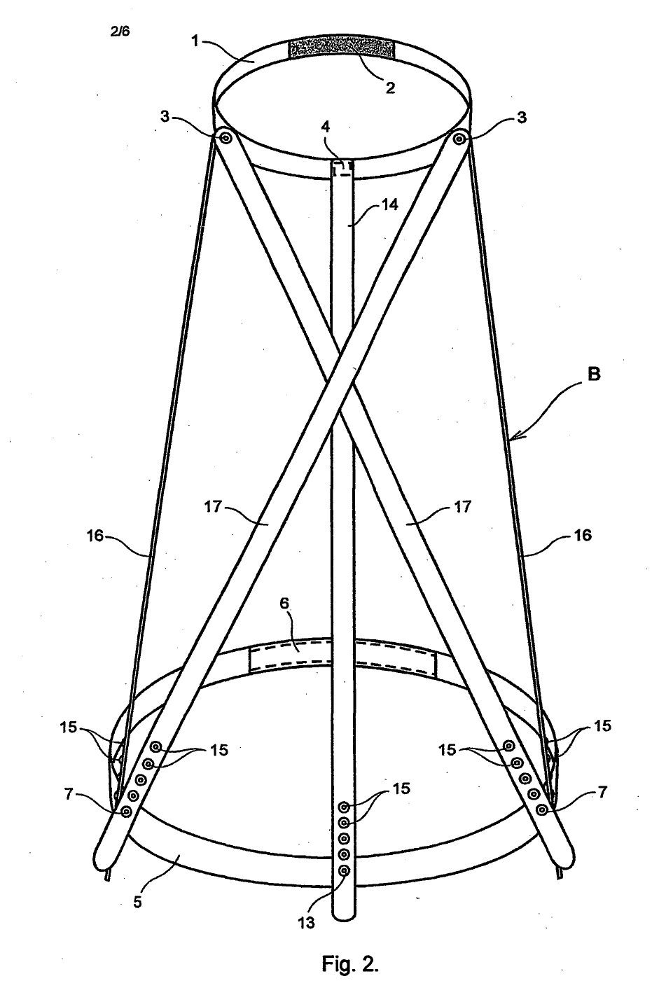

### Corset B, FIG. 2, Comprises:

head strap 1, with fasteners 3, for detaching and connecting strips 16 and 17;
waist strap 5, with fasteners 7 and 13, for detaching and connecting strips 16 and 17;
strip 14, with holes 15 for detaching and connecting to fastener 13; and
strips 16 and 17, with holes 15, for detaching and connecting to fastener 7.

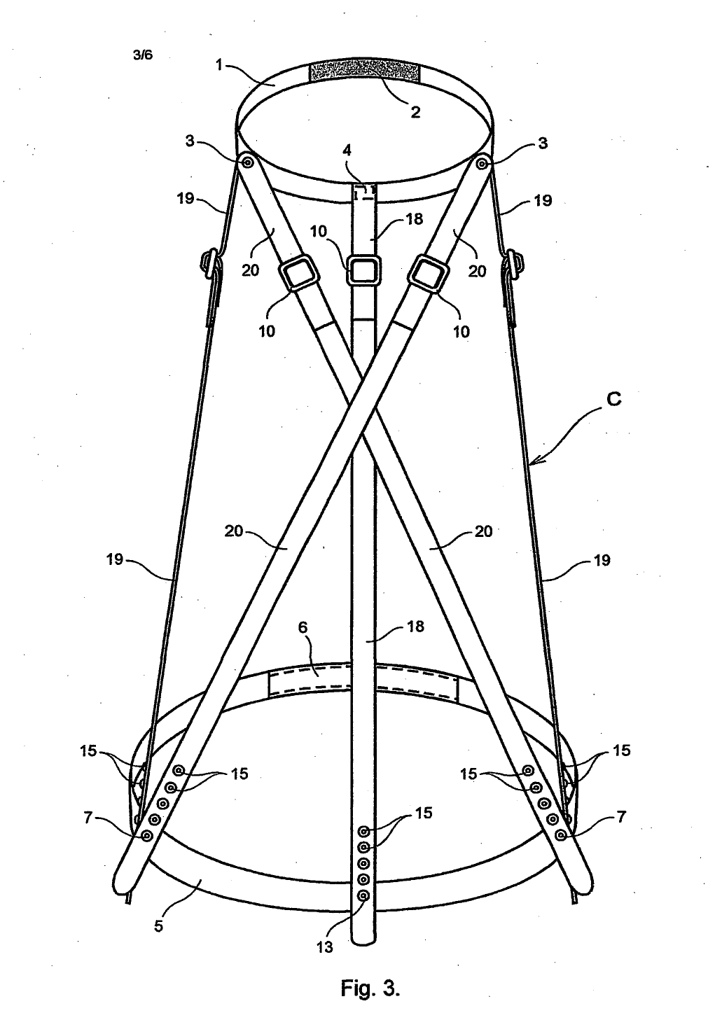

### Corset C, FIG. 3, Comprises:

head strap 1, with fasteners 3, for detaching and connecting strips 19 and 20;
waist strap 5, with fasteners 7 and 13, for detaching and connecting strips 18, 19, and 20;
strips 18, 19, and 20, with buckles 10 and holes 15, for connecting to fasteners 7 and 13; and
strips 20, crossed over the back.

Strap 1 is fitted to the user's head, and fixed with velcro strap 2, whereas strap 5 is fitted around the waist and fixed with velcro strap 6.

Strips 9, 11, and 12 in the corset A embodiment, and strips 18, 19, and 20 in the corset C embodiment are made of two parts, mutually connected with buckles 10 that enables adjustment in length. Holes 15 in the lower part of the strips enable adjustment of the strips to the user's needs.

Strips 9, 14, and 18 are mounted across the back, to limit the head from bending forward. Strips 11, 16, and 19 limit the head from bending to the right and left sides. Strips 12, 17, and 20 are crossed to limit rotation of the head around the vertical axis.

Fasteners 3 are fixed to the head strap 1 at its opposite sides.

Fasteners 7 are fixed to the waist strap 5 at its opposite sides.

Fastener 13 is fixed to the middle of the back part of the waist strap.

Fasteners 3, 7, and 13, with one or more openings 15 and buckles 10, may be embodied in any of the already known ways.

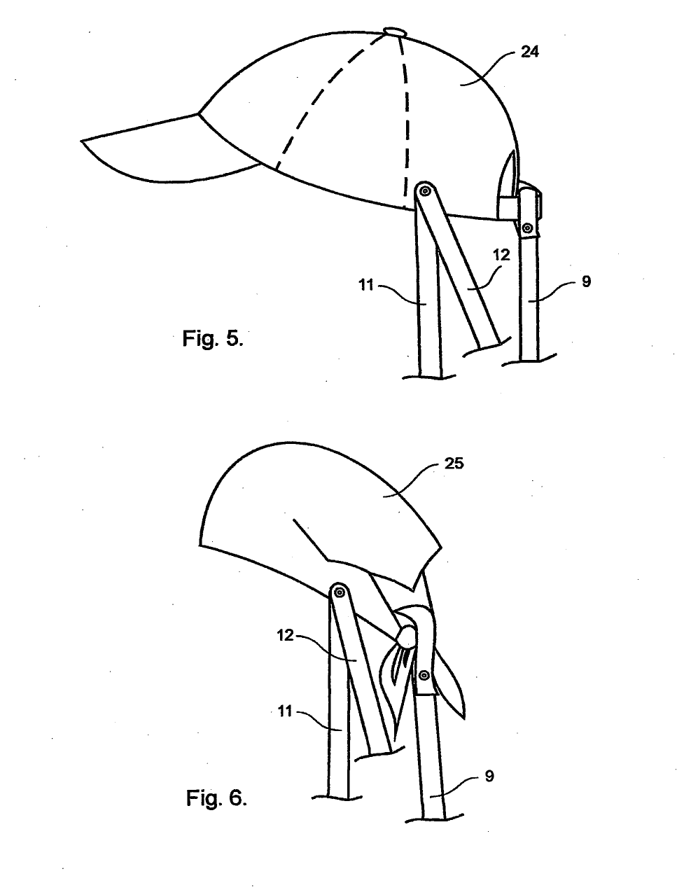

### Cap Functioning as the Head Strap, FIG. 5

FIG. 5 shows the strips 9, 11, and 12 fitted to a cap 24 to limit the head from bending and rotating. The cap may be of any shape, however, its function, with fitted strips 9, 11, and 12, will remain unchanged. The strips 9, 11, and 12 may be fixed to a cap in any of the already known ways.

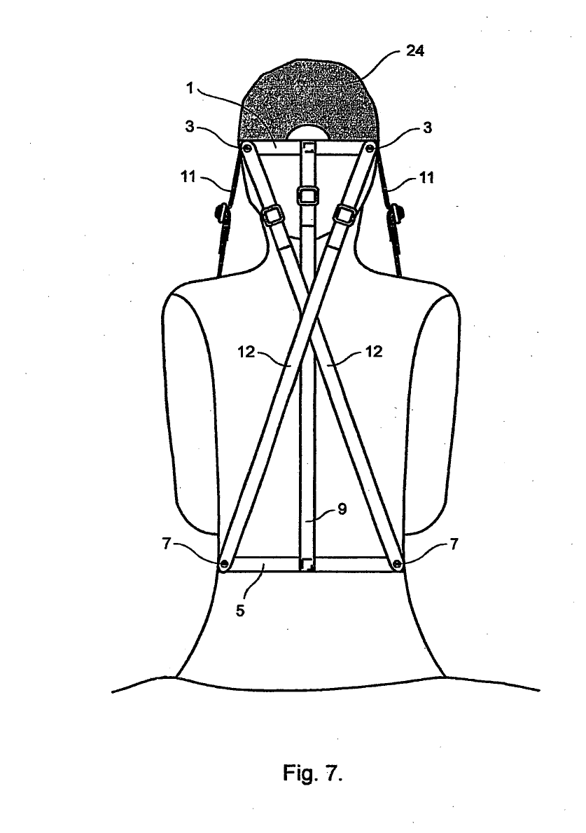

### Head Scarf Functioning as the Head Strap, FIG. 6

FIG. 6 shows the strips 9, 11, and 12 fitted to a head scarf 25 to limit the head from bending and rotating. The scarf may be of any shape, however, its function, with fitted strips 9, 11, and 12, will remain unchanged. The strips 9, 11, and 12 may be fixed to a head scarf in any of the already known ways.

### Functioning of the Corset

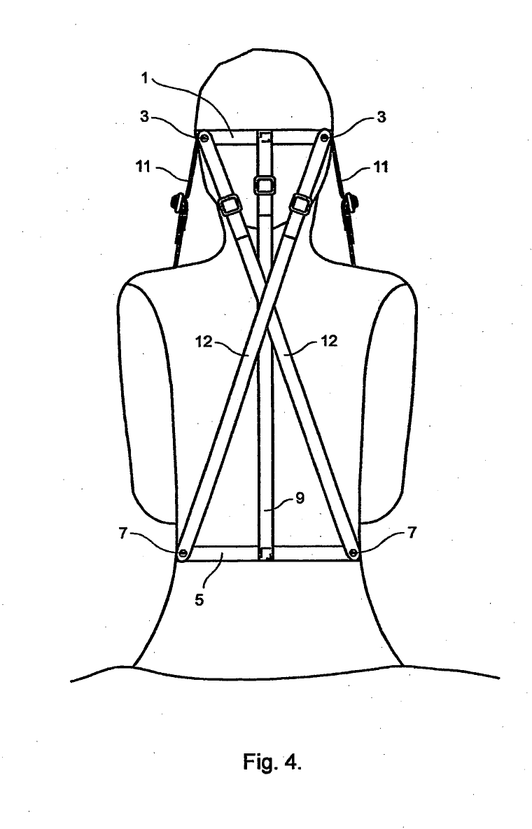

The corset that relieves the cervical spine is fitted around the head and the waist, as shown in FIGS. 4 and 7. Any of the three corset embodiments can be used depending on whether the head strap 1 is applied directly to the head, a cap 24, or a head scarf 25. The head strap 1 is fixed over the forehead and the waist strap 5 around the body in the lumbar region. After this, and depending on the discomfort, an adequate strip or strips are fitted and adjusted.

The corset that relieves the cervical spine functions when its user, by their movements, brings the cervical spine into an unfavourable position. This results in a significant fixation that enhances effects of eccentric contraction of the deep neck muscles. This bending and rotating of the head can significantly diminish aggravating effects to the cervical spine.

In such situations, strips 9, 11, and 12 in the corset A embodiment, strips 14, 16, and 17 in the corset B embodiment, or strips 18, 19, and 20 in the corset C embodiment stretch and prevent overloading of the neck and damages to the discs. Simultaneously, the eccentric contraction of the neck muscles is supported, which decreases fatigue which, in turn, enables longer performance of jobs that cause the neck to bend. Furthermore, in damaged cervical spines, adjustment of strips 9, 11, and 12 in the corset A embodiment, strips 14, 16, and 17 in the corset B embodiment, or strips 18, 19, and 20 in the corset C embodiment enables blocking that very movement that aggravates the situation.

What is claimed is:

1. A corset for a human user to relieve the cervical spine, the corset comprising:
a head strap;
a waist strap with said head strap and said waist strap being mutually connected with one strip to limit forward movement of the user's head;
two additional strips with each strip being connected between said head strap and said waist strap; each said additional strip limiting the sideward movement of the user's head about an imaginary vertical axis through the user's head; and
wherein said three strips are crossed over about the user's back.

2. The corset according to claim 1 wherein the strips are adjustable in length.

3. The corset according to claim 1 wherein the head strap further comprises a cap.

4. The corset according to claim 1 wherein the head strap further comprises a head scarf.

5. The corset according to claim 1 wherein the head strap has hook and loop fasteners for adjusting and tightening the strap about the user's head.

6. The corset according to claim 1 wherein the three strips are moveably fixed to the waist strap.

7. The corset according to claim 2 wherein each strip further comprises at least one buckle.

8. The corset according to claim 2 wherein each strip further comprises a plurality of holes.

9. The corset according to claim 2 wherein each strip further comprises at least one buckle and a plurality of holes.

10. The corset according to claim 3 wherein the cap has hook and loop fasteners for adjusting and tightening the strap about the user's head.

11. A corset for a human user to relieve the cervical spine, the corset comprising:
a head strap;
a waist strap with said head strap and said waist strap being mutually connected with one strip to limit forward movement of the user's head;
two additional strips with each strip being connected between said head strap and said waist strap; each said additional strip limiting the sideward movement of the user's head about an imaginary vertical axis through the user's head; and
wherein said three strips are crossed over about the user's back and are of adjustable length;
and wherein the three strips are moveably fixed to the waist strap.

12. The corset according to claim 11 wherein each strip further comprises at least one buckle.

13. The corset according to claim 11 wherein each strip further comprises a plurality of holes.

14. The corset according to claim 11 wherein each strip further comprises at least one buckle and a plurality of holes.

15. A corset that relieves the cervical spinecomprising:
a head strap (1) and a waist strap (5), where, with regard to the construction and ways of connecting of strips to the head strap and the waist strap there are three corset embodiments: corset (A) with rings on the strips, corset (B) with holes on the strips, and corset (C) with rings and holes on the strips;
wherein the corset (A) the head strap and the waist strap are connected with the strip (9) passing over the back and limiting bending of the head forward, at lateral sides with the strips (11) limiting bending of the head sideward, and at lateral sides with the strips (12) limiting the head rotation around its vertical axis;
wherein the corset (B) the head strap and the waist strap are connected with the strip (14) passing over the back and limiting bending of the head forward, at lateral sides with the strips (16) limiting bending of the head sideward, and at lateral sides with the strips (17) limiting the head rotation around its vertical axis;
wherein the corset (C) the head strap and the waist strap are connected with the strip (18) passing over the back and limiting bending of the head forward, at lateral sides with the strips (19) limiting bending of the head sideward, and at lateral sides with the strips (20) limiting the head rotation around its vertical axis;
wherein the strips (9, 11, and 12) on the corset (A), and the strips (18, 19, and 20) on the corset (C) are made of two parts with rings (10) for adjusting the length;
wherein the case of corset (A) the strip (9) is fixed firmly with one end in the position (4) to the head strap (1) and with the other end in the position (8) to the waist strap (5), the strips (11 and 12) are fixed with fasteners (3) and are fixed movably to the head strap and with the other end via the fasteners (7) that are fixed movably to the waist strap, and the strips (12) are crossed over the back;
wherein the case of corset (B) the strip (14) is firmly fixed with one end to the head strap and with the other end by holes (15) and a fastener (13) that is fixed movably to the waist strap, the strips (16 and 17) are fixed movably by fasteners (3) at one end to the head strap, and with the other end through the holes (15) and by fasteners (7) that are movably fixed to the waist strap, and the strips (17) are crossed over the back;
wherein the case of corset (C) the strip (18) is fixed firmly to the head strap at one end and the other end with the holes (15) and by a fastener (13) that is fixed movably to the waist strap, and the strips (19 and 20) are fixed movably at one end with a fastener (3) to the head strap and the other end with holes (15) and are fixed movably by a fastener (7) to the waist strap and the strips (20) are crossed over the back.

16. The corset that relieves the cervical spine according to claim 15 wherein the head strap (1) has a hook and loop fastener strap (2) for adjusting and tightening the strap around the user's head.

17. The corset that relieves the cervical spine according to claim 15 wherein the waist strap (5) has a hook and loop fastener strap (6) for adjusting and tightening the strap around the user's waist.

18. The corset that relieves the cervical spine according to claim 15, wherein the fasteners (3) are fixed to the outer side of the head strap (1) at its opposite sides.

19. The corset that relieves the cervical spine according to claim 15, wherein the fastener (13) is fixed in the middle of the back part of the waist strap (5).

20. The corset that relieves the cervical spine according to claim 15, wherein the fasteners (7) are fixed to the waist strap (5) at its opposite sides.

21. The corset that relieves the cervical spine according to claim 15, wherein the strips (14, 16, 17, 18, 19, and 20) have in their lower part at least one hole (15) that enables adjustment of length of the arms when fitted to the fasteners (7 and 13) on the waist strap.
